(19) United States
(12) Patent Application Publication
(10) Pub. No.: US 2025/0264459 A1
(43) Pub. Date: Aug. 21, 2025

(54) IN VITRO ASSAY TO PREDICT CARDIOTOXICITY

(71) Applicant: Stemina Biomarker Discovery, Inc., Madison, WI (US)

(72) Inventors: Jessica A. Palmer, Madison, WI (US); Alan M. Smith, Madison, WI (US); Robert Burriere, Verona, WI (US)

(21) Appl. No.: 18/884,815

(22) Filed: Sep. 13, 2024

Related U.S. Application Data
(63) Continuation of application No. 16/977,970, filed on Sep. 3, 2020, now abandoned.
(60) Provisional application No. 62/640,722, filed on Mar. 9, 2018.

(51) Int. Cl. G01N 33/50

(52) U.S. Cl. CPC G01N 33/5061; G01N 33/5014

(57) ABSTRACT

The invention provides methods for predicting whether compounds are cardiotoxic by analyzing their effects on the ratios of concentrations of metabolites in cultured heart muscle cells.

### CROSS-REFERENCE TO RELATED APPLICATIONS

This application claims the benefit of, and priority, U.S. Provisional Application No. 62/640,722, filed Mar. 9, 2018, the contents of which are incorporated herein by reference.

### FEDERAL GRANT FUNDING

This invention was made with government support under Grant No. 2R440M100640-02, awarded by that National Institutes of Health. The Government has certain rights in the invention.

### BACKGROUND

Cardiotoxicity is a risk associated with many licensed drugs and is the cause of failure of many other drug candidates during clinical trials.

### SUMMARY

The invention provides methods for predicting whether compounds are cardiotoxic by analyzing their effects on the ratios of concentrations of metabolites in cultured heart muscle cells.

In some aspects, the method includes removing proteins from the sample of the spent cell culture media prior to assaying the sample of the spent cell culture media for one or more small molecule metabolites. In some aspects, proteins may be removed from the sample of spent cell culture media precipitation. In some aspects, precipitation may be at about 50% methanol, about 35% acetonitrile, and about 15% sample.

In some aspects, assaying includes assaying for two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, or all thirty one of the small molecule metabolites listed in Table 2 of Example 2.

In some aspects, assaying includes assaying for one or more, two or more, three or more, four or more, five or more, six or more, seven or more, or all eight of arachidonic acid, thymidine, pyruvate, 2′-deoxycytidine, inosine, lactic acid, alanine, and N-acetylaspartic acid.

In some aspects, assaying includes assaying for lactic acid, thymidine, arachidonic acid, 2-deoxycytidine, and N-acetylaspartic acid.

In some aspects, assaying includes assaying for alanine, pyruvate, and inosine.

In some aspects, the method further includes an assessment of the relative abundance of two or more of the small molecule metabolites.

In some aspects, the method further includes determining a ratio of two or more of the small molecule metabolites.

In some aspects, the method further includes assaying for lactic acid and arachidonic acid; 2-deoxycytidine and thymidine; lactic acid and thymidine; and/or N-acetylaspartate and 2′-deoxycytidine.

In some aspects, the method further includes determining the ratio of lactic acid to arachidonic acid; 2′-deoxycytidine to thymidine; lactic acid to thymidine; and/or N-acetylaspartate to 2′-deoxycytidine. In some aspects, the ratio of lactic acid to arachidonic acid, 2′-deoxycytidine to thymidine, lactic acid to thymidine, and/or N-acetylaspartate to 2′-deoxycytidine is indicative of cardiotoxicity. In some aspects, a ratio of lactic acid to arachidonic acid of about 0.84 or less is indicative of cardiotoxicity.

In some aspects, the method further includes determining two or more ratios of small molecule metabolites. In some aspects, the method includes determining the ratios of lactic acid to thymidine and N-acetylaspartate to 2′-deoxycytidine; lactic acid to thymidine and 2′-deoxycytidine to thymidine; arachidonic acid to lactic acid and N-acetylaspartate to 2′-deoxycytidine; and/or arachidonic acid to lactic acid and 2′-deoxycytidine to thymidine. In some aspects, one or more pairings of ratios is indicative of cardiotoxicity.

The present invention includes a method including contacting an in vitro culture of human cardiomyocyte cells with a test compound; and assaying the spent cell culture media for one or more of the small molecule metabolites listed in Table 2 of Example 2.

In some aspects, the method includes removing proteins from the sample of the spent cell culture media prior to assaying a sample of the spent cell culture media for the one or more of the small molecule metabolites. In some aspects, proteins may be removed from the sample of spent cell culture media precipitation. In some aspects, precipitation may be at about 50% methanol, about 35% acetonitrile, and about 15% sample.

In some aspects, assaying includes assaying for two or more, three or more, four or more, live or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, or all thirty one of the small molecule metabolites listed in Table 2 of Example 2.

In some aspects, assaying includes assaying for one or more, two or more, three or more, four or more, five or more, six or more, seven or more, or all eight of arachidonic acid, thymidine, pyruvate, 2′-deoxycytidine, inosine, lactic acid, alanine, and N-acetylaspartic acid.

In some aspects, assaying includes assaying for lactic acid, thymidine, arachidonic acid, 2′-deoxycytidine, and N-acetylaspartic acid.

In some aspects, assaying includes assaying for alanine, pyruvate, and inosine.

In some aspects, the method further includes an assessment of the relative abundance of two or more small molecule metabolites.

In some aspects, the method further includes determining a ratio of two or more small molecule metabolites.

In some aspects, the method further includes assaying for lactic acid and arachidonic acid; 2′-deoxycytidine and thymidine; lactic acid and thymidine; and/or N-acetylaspartate and 2′-deoxycytidine.

In some aspects, the method further includes determining the ratio of lactic acid to arachidonic acid; 2′-deoxycytidine to thymidine; lactic acid to thymidine; and/or N-acetylaspartate to 2′-deoxycytidine.

In some aspects, the method further includes determining two or more ratios of the small molecule metabolites.

In some aspects, the method further includes determining the ratios of: lactic acid to thymidine and N-acetylaspartate to 2′-deoxycytidine; lactic acid to thymidine and 2′-deoxycytidine to thymidine; arachidonic acid to lactic acid and N-acetylaspartate to 2′-deoxycytidine; and/or arachidonic acid to lactic acid and 2′-deoxycytidine to thymidine.

With any of the methods described herein, the one or more the small molecule metabolites may be assayed by a physical separation method. In some aspects, a physical separation method includes liquid chromatography mass spectrometry (LC-MS). In some aspects, a physical separation method includes one or more methodologies selected from CS liquid chromatography coupled to electrospray ionization in positive ion polarity (C8pos). C8 liquid chromatography coupled to electrospray ionization in negative ion polarity (C8neg), hydrophilic interaction liquid chromatography coupled to electrospray ionization in positive ion polarity (HILICpos), and/or hydrophilic interaction liquid chromatography coupled to electrospray ionization in negative ion polarity (HILICneg).

With any of the methods described herein, the one or more the small molecule metabolites may be assayed using a colorimetric or immunological assay.

With any of the methods described herein, the cardiomyocytes may be cultured at a concentration of the test compound comprising the test compound's human therapeutic Cₘₐₓ.

With any of the methods described herein, human cardiomyocyte cells include comprise human induced pluripotent stem cell (hiPSC)-derived cardiomyocyte cells.

The present invention includes a method of assessing cardiotoxicity of a test compound as described herein.

The present invention includes a metabolomic signature for cardiotoxicity including two or more of the features set forth in Table 2 of Example 2.

The present invention includes a metabolomic signature for cardiotoxicity including two or more, three or more, four or more, five or more, six or more, seven or more, or all eight of arachidonic acid, thymidine, pyruvate, 2′-deoxycytidine, inosine, lactic acid, alanine, and N-acetylaspartic acid.

The present invention includes a metabolomic signature for cardiotoxicity as described herein.

### BRIEF DESCRIPTION OF THE DRAWINGS

FIG. 1 is a schematic of the assay development workflow in an embodiment of the invention.

FIG. 2 is a graph showing the interpolated response at the Cₘₐₓ classification exposure level from the dose-response model fit of a ratio of the fold changes of lactic acid and arachidonic acid normalized to viability.

FIG. 3 is a schematic of the method used to evaluate cardiotoxicity of compounds.

FIG. 4 is a schematic of the assay development workflow used to evaluate cardiotoxicity of compounds.

FIG. 5 shows graphs from results of the concentration-response phase of biomarker identification.

FIG. 6 is a table showing exposure levels selected for single exposure phase.

FIG. 7 is a table showing training set results from the single exposure phase.

FIG. 8 is a table of non-cardiotoxicants and cardiotoxicants identified in the single exposure phase.

FIG. 9 is a table showing characteristics of various global toxicity models.

FIG. 10 shows training set results for individual metabolites and metabolite condition utilized in the Cardio quickPredict assay at the therapeutic total Cₘₐₓ, and 10×Cₘₐₓ.

FIG. 11 shows graphs of the metabolite responses for a subset of the training set compounds.

FIG. 12 is a table of compounds tested in the single exposure phase and whether they were predicted to be cardiotoxicants by Cardio quickPredict.

### DETAILED DESCRIPTION

Cardiac safety is one of the leading causes of late-stage compound attrition in the pharmaceutical industry and accounts for 28% of the safety related withdrawals of FDA-approved drugs from the market. Current cardiac safety preclinical evaluations are heavily focused on approximately 3-7 main ion channels involved in maintaining the cardiac action potential. However, over 70 different types of ion channels are expressed in the heart and participate in the overall cardiac current. These safety testing methods overemphasize electrophysiological assessment of cardiotoxicity and fail to evaluate cardiomyopathy and other forms of structural cardiotoxicity. Thus, there is a need for improved methods for the identification of cardiotoxicity of pharmaceuticals.

Metabolic perturbations are one of the primary mechanisms underlying the cardiotoxicity elicited by pharmaceuticals. With this invention, a small molecule biomarker-based assay has been developed for evaluating the cardiotoxicity potential of compounds based on changes in the metabolism and viability of cardiomyocytes cultured in vitro with the compound. Metabolomic analysis of spent media identified a set of predictive biomarkers. This assay is an attractive new model that can identify both structural and functional cardiotoxic compounds that could be used in conjunction with CiPA and other endpoints to provide a more comprehensive evaluation of a compound's cardiotoxicity potential.

For example, as described in the examples included herewith herein, hiPSC-derived cardiomyocytes were exposed to a training set of 57 compounds (37 positive, 20 negative) and blinded test set of 12 compounds (6 positive, 6 negative). The cardiotoxic compounds were broken into three categories, structural, functional, and general (compounds that cause both). Metabolomic analysis of spent media identified a set of predictive biomarkers. The biomarker-based model classified the test set with 92% accuracy and the training set with 86% accuracy, based on comparing the concentration where metabolism was perturbed to the therapeutic Cₘₐₓ. This assay is an attractive new model that can identify both structural and functional cardiotoxic compounds that could be used in conjunction with CiPA and other endpoints to provide a more comprehensive evaluation of a compound's cardiotoxicity potential.

The present invention includes a method of assessing cardiotoxicity of a test compound, the method including contacting an in vitro culture of human cardiomyocyte cells with a test compound; assaying the spent cell culture media for one or more of the small molecule metabolites listed in Table 2 of Example 2; wherein a statistically significant abundance difference as compared to cardiomyocyte controls in the one or more small molecule metabolites indicates cardiotoxicity.

In some aspects, the method includes removing proteins from the sample of the spent cell culture media prior to assaying the sample of the spent cell culture media for one or more small molecule metabolites. In some aspects, proteins may be removed from the sample of spent cell culture media precipitation. In some aspects, precipitation may be at about 50% methanol, about 35% acetonitrile, and about 15% sample.

In some aspects, assaying includes assaying for two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, or all thirty one of the small molecule metabolites listed in Table 2 of Example 2.

In some aspects, assaying includes assaying for one or more, two or more, three or more, four or more, five or more, six or more, seven or more, or all eight of arachidonic acid, thymidine, pyruvate, 2′-deoxycytidine, inosine, lactic acid, alanine, and N-acetylaspartic acid.

In some aspects, assaying includes assaying for lactic acid, thymidine, arachidonic acid, 2′-deoxycytidine, and N-acetylaspartic acid.

In some aspects, assaying includes assaying for alanine, pyruvate, and inosine.

In some aspects, the method further includes an assessment of the relative abundance of two or more of the small molecule metabolites.

In some aspects, the method further includes determining a ratio of two or more of the small molecule metabolites.

In some aspects, the method further includes assaying for lactic acid and arachidonic acid; 2′-deoxycytidine and thymidine; lactic acid and thymidine; and/or N-acetylaspartate and 2′-deoxycytidine.

In some aspects, the method further includes determining the ratio of lactic acid to arachidonic acid; 2′-deoxycytidine to thymidine; lactic acid to thymidine; and/or N-acetylaspartate to 2′-deoxycytidine. In some aspects, the ratio of lactic acid to arachidonic acid, 2′-deoxycytidine to thymidine, lactic acid to thymidine, and/or N-acetylaspartate to 2′-deoxycytidine is indicative of cardiotoxicity. In some aspects, a ratio of lactic acid to arachidonic acid of about 0.84 or less is indicative of cardiotoxicity.

In some aspects, the method further includes determining two or more ratios of small molecule metabolites. In some aspects, the method includes determining the ratios of lactic acid to thymidine and N-acetylaspartate to 2′-deoxycytidine; lactic acid to thymidine and 2′-deoxycytidine to thymidine; arachidonic acid to lactic acid and N-acetylaspartate to 2′-deoxycytidine; and/or arachidonic acid to lactic acid and 2′-deoxycytidine to thymidine. In some aspects, one or more pairings of ratios is indicative of cardiotoxicity.

The present invention includes a method including contacting an in vitro culture of human cardiomyocyte cells with a test compound; and assaying the spent cell culture media for one or more of the small molecule metabolites listed in Table 2 of Example 2.

In some aspects, the method includes removing proteins from the sample of the spent cell culture media prior to assaying a sample of the spent cell culture media for the one or more of the small molecule metabolites. In some aspects, proteins may be removed from the sample of spent cell culture media precipitation. In some aspects, precipitation may be at about 50% methanol, about 35% acetonitrile, and about 15% sample.

In some aspects, assaying includes assaying for two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, or all thirty one of the small molecule metabolites listed in Table 2 of Example 2.

In some aspects, assaying includes assaying for one or more, two or more, three or more, four or more, five or more, six or more, seven or more, or all eight of arachidonic acid, thymidine, pyruvate, 2′-deoxycytidine, inosine, lactic acid, alanine, and N-acetylaspartic acid.

In some aspects, assaying includes assaying for lactic acid, thymidine, arachidonic acid, 2′-deoxycytidine, and N-acetylaspartic acid.

In some aspects, assaying includes assaying for alanine, pyruvate, and inosine.

In some aspects, the method further includes an assessment of the relative abundance of two or more small molecule metabolites.

In some aspects, the method further includes determining a ratio of two or more small molecule metabolites.

In some aspects, the method further includes assaying for lactic acid and arachidonic acid; 2′-deoxycytidine and thymidine; lactic acid and thymidine; and/or N-acetylaspartate and 2′-deoxycytidine.

In some aspects, the method further includes determining the ratio of lactic acid to arachidonic acid; 2′-deoxycytidine to thymidine; lactic acid to thymidine; and/or N-acetylaspartate to 2′-deoxycytidine.

In some aspects, the method further includes determining two or more ratios of the small molecule metabolites.

In some aspects, the method further includes determining the ratios of: lactic acid to thymidine and N-acetylaspartate to 2′-deoxycytidine; lactic acid to thymidine and 2′-deoxycytidine to thymidine; arachidonic acid to lactic acid and N-acetylaspartate to 2′-deoxycytidine; and/or arachidonic acid to lactic acid and 2′-deoxycytidine to thymidine.

With any of the methods described herein, the one or more the small molecule metabolites may be assayed by a physical separation method. In some aspects, a physical separation method includes liquid chromatography mass spectrometry (LC-MS). In some aspects, a physical separation method includes one or more methodologies selected from C8 liquid chromatography coupled to electrospray ionization in positive ion polarity (C8pos), C8 liquid chromatography coupled to electrospray ionization in negative ion polarity (C8neg), hydrophilic interaction liquid chromatography coupled to electrospray ionization in positive ion polarity (HILICpos), and/or hydrophilic interaction liquid chromatography coupled to electrospray ionization in negative ion polarity (HILICneg).

With any of the methods described herein, the one or more the small molecule metabolites may be assayed using a colorimetric or immunological assay.

With any of the methods described herein, the cardiomyocytes may be cultured at a concentration of the test compound comprising the test compound's human therapeutic Cₘₐₓ.

With any of the methods described herein, human cardiomyocyte cells include comprise human induced pluripotent stem cell (hiPSC)-derived cardiomyocyte cells.

The present invention includes a method of assessing cardiotoxicity of a test compound as described herein.

The present invention includes a metabolomic signature for cardiotoxicity including two or more of the features set forth in Table 2 of Example 2.

The present invention includes a metabolomic signature for cardiotoxicity including two or more, three or more, four or more, five or more, six or more, seven or more, or all eight of arachidonic acid, thymidine, pyruvate, 2′-deoxycytidine, inosine, lactic acid, alanine, and N-acetylaspartic acid.

The present invention includes a metabolomic signature for cardiotoxicity as described herein.

Cardiomyocyte or cardiomyocyte cell(s) may include primary cardiomyocytes, cardiomyocyte precursor cells, clonal cardiomyocytes derived from adult human heart, immortalized cardiomyocytes, human embryonic stem cell (hESC)-derived cardiomyocytes, human induced pluripotent stem cell (iPS)-derived cardiomyocytes, or any cell displaying cardiomyocyte-specific markers such that a pathologist, scientist, or laboratory technician would recognize the cell to be cardiomyocyte-specific or cardiomyocyte derived.

Cardiotoxic or cardiotoxicity refers to a substance or treatment, particularly pharmaceuticals, biologics, and other chemical compounds and environmental agents, that induce cardiomyopathy, heart disease, and/or abnormal heart pathology and physiology. Examples of cardiotoxicities encompassed by the definition of the term as used herein include heart abnormalities that would be recognized by a physician, cardiologist, or medical researcher, which could be attributed to or a potential result of a drug-treatment regimen.

In a preferred embodiment the term “compound” or “test compound” includes but is not limited to pharmaceuticals, environmental agents, chemical compounds and biologic therapies, including antibody-based treatments, vaccines, or recombinant proteins and enzymes. In a particularly preferred embodiment, cardiotoxic compounds include tamoxifen, doxorubicin, and paclitaxel. In a further embodiment, potentially cardiotoxic compounds are screened for metabolite similarities to already known cardiotoxic compounds.

In some embodiments, alterations in small molecule metabolites arc measured by determining changes in treated versus untreated cells. Also included are comparisons between cells treated with different amounts, types or concentrations, durations or intensities of cardiotoxic or potential cardiotoxic compounds.

In some aspects, a given small molecule metabolite is produced greater amounts in cardiomyocytes contacted with the test compound.

In some aspects, a given small molecule metabolite is produced in greater amounts in cardiomyocytes not contacted with the test compound.

Small molecule metabolites may be detected, identified, assayed, quantified by any of a variety of methods. For example, small molecule metabolites may assayed using a physical separation method, such as, for example, one or more methodologies selected from gas chromatography mass spectrometry (GCMS), C8 liquid chromatography coupled to electrospray ionization in positive ion polarity (C8pos), C8 liquid chromatography coupled to electrospray ionization in negative ion polarity (C8neg), hydrophilic interaction liquid chromatography coupled to electrospray ionization in positive ion polarity (HILICpos), and/or hydrophilic interaction liquid chromatography coupled to electrospray ionization in negative ion polarity (HILICneg).

Small molecule metabolites may be identified by their unique molecular mass and consistency, thus the actual identity of the underlying compound that corresponds to the biomarker is not required for the practice of this invention. Biomarkers may be identified using, for example, Mass Spectrometry such as MALDI/TOF (time-of-flight). SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, tandem mass spectrometry (e.g., MS/MS, MS/MS/MS. ESI-MS/MS etc.), secondary ion mass spectometry (SIMS), and/or ion mobility spectrometry (e.g. GC-IMS, IMS-MS, LC-IMS, LC-IMS-MS etc.).

Small molecule metabolites can be detected using any of the methods described herein. Metabolites, as set forth herein, can be detected using alternative spectrometry methods or other methods known in the art, in addition to any of those described herein. In some aspects, the determination of a metabolite may be by a methodology other than a physical separation method, such as for example, a colorimetric, enzymatic, immunological methodology, and gene expression analysis, including, for example, real-time PCR, RT-PCR. Northern analysis, and in situ hybridization.

With the preparation of samples for analysis, metabolites may be extracted from culture media using any number of extraction/clean-up procedures that are typically used in quantitative analytical chemistry. In some aspects, proteins may be removed, for example, by precipitation. In some aspects, protein precipitation is with about 50% methanol, about 35% acetonitrile, and about 15% sample.

The small molecule metabolites described herein may be utilized in tests, assays, methods, kits for diagnosing, predicting, modulating, or monitoring cardiotoxicity. The present invention includes a kit for identifying and/or measuring one or more metabolites associated with cardiotoxicity. In some aspects, the kit may be for the determination of a metabolite by a physical separation method. In some aspects, the kit may be for the determination of a metabolite by a methodology other than a physical separation method, such as for example, a colorimetric, enzymatic, immunological methodology. In some aspects an assay kit may also include one or more appropriate negative controls and/or positive controls. Kits of the present invention may include other reagents such as buffers and solutions needed to practice the invention are also included. Optionally associated with such container(s) can be a notice or printed instructions. As used herein, the phrase “packaging material” refers to one or more physical structures used to house the contents of the kit. The packaging material is constructed by well-known methods, preferably to provide a sterile, contaminant-free environment. As used herein, the term “package” refers to a solid matrix or material such as glass, plastic, paper, foil, and the like. Kits of the present invention may also include instructions for use. Instructions for use typically include a tangible expression describing the reagent concentration or at least one assay method parameter, such as the relative amounts of reagent and sample to be admixed, maintenance time periods for reagent/sample admixtures, temperature, buffer conditions, and the like. In some aspects, a kit may be a packaged combination including the basic elements of a first container including, in solid form, a specific set of one or more purified metabolites, as described herein, and a second container including a physiologically suitable buffer for resuspending or dissolving the specific subset of purified metabolites. One or more of the metabolites described herein may be present in a kit.

The term “and/or” means one or all of the listed elements or a combination of any two or mom of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also, recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples am reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### In Vitro Assay to Predict Cardiotoxicity Potential Using Targeted Metabolomics and Human Induced Pluripotent Stem Cell-Derived Cardiomyocytes

Cardiac safety is one of the leading causes of late-stage compound attrition in the pharmaceutical industry and accounts for 28% of the safety related withdrawals of FDA-approved drugs from the market. Current cardiac safety preclinical evaluations are heavily focused on approximately 3-7 main ion channels involved in maintaining the cardiac action potential; however, over 70 different types of ion channels are expressed in the heart and participate in the overall cardiac current. These safety testing methods overemphasize electrophysiological assessment of cardiotoxicity and fail to evaluate cardiomyopathy and other forms of structural cardiotoxicity. Metabolic perturbations are one of the primary mechanisms underlying the cardiotoxicity elicited by pharmaceuticals. With this invention, a small molecule biomarker-based assay has been developed for evaluating the cardiotoxicity potential of compounds based on changes in the metabolism and viability of human induced pluripotent stem cell (hiPSC)-derived cardiomyocytes. As described herein, hiPSC-derived cardiomyocytes were exposed to a training set of 57 compounds (37 positive, 20 negative) and blinded test set of 12 compounds (6 positive, 6 negative). The cardiotoxic compounds were broken into three categories, structural, functional, and general (compounds that cause both). Metabolomic analysis of spent media identified a set of predictive biomarkers. The biomarker-based model classified the test set with 92% accuracy and the training set with 86% accuracy, based on comparing the concentration where metabolism was perturbed to the therapeutic Cₘₐₓ. This assay is an attractive new model that can identify both structural and functional cardiotoxic compounds that could be used in conjunction with CiPA and other endpoints to provide a more comprehensive evaluation of a compound's cardiotoxicity potential.

### Example 2

### Development of LC-MS-Based Platform for Analysis of Spent Medium Collected from Cardiomyocytes

Specific Aim 1: To develop highly reproducible LC-MS methods and increase the number of secreted features measured in spent cell culture media from human induced pluripotent stem cell-derived cardiomyocytes.

A “mass feature” (also referred to as a “feature”) is: 1) a molecule detected by the mass spectrometer with a measured abundance, 2) a detected mass-to-charge ratio (m/z) and 3) a chromatographic retention time. Briefly, the first step in developing the LC-MS methods was to develop a new method for sample preparation to remove proteins from the samples. Of the 14 sample preparation methods evaluated, a protein precipitation method using methanol and acetonitrile (50% methanol, 35% acetonitrile, 15% sample) was selected. This method was the most effective at removing proteins from the sample while maintaining acceptable recovery levels for the metabolites evaluated.

A total of 11 different LC-MS methods were tested that included four HPLC columns with different gradients in positive and negative electrospray ionization modes to increase the reproducibility and number of features measured. Of these, four chromatographic separation methods (two columns. HILIC and C8, two ionization modes each) were carried forward from method development into robustness testing. The robustness and reproducibility of the chromatography methods was evaluated using three lots of each column (HILIC and C8) in two sample sets, i.e. two cell culture plates were analyzed in each ionization mode on each column lot. All four methods passed robustness testing and were used for the discovery experiments in Aim 2. With these four methods, we identified 176 mass features associated with the human iPS cell-derived cardiomyocyte (iPSC-CM) secretome (small molecules that are secreted and/or consumed by cells).

Specific Aim 2: Acquire and analyze metabolomic data from iPS cell-derived cardiomyocytes exposed to a training set of known human cardiotoxicants.

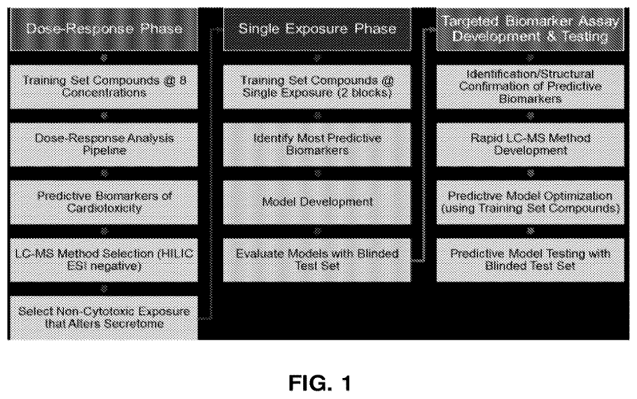

The objective of Aim 2 was to identify differential metabolites and a predictive metabolic signature indicative of cardiotoxicity. To accomplish this, two experiments were performed that consisted of three independent experiments using the training set (Table 1, FIG. 1).

FIG. 1 is a schematic of the assay development workflow. In the first experiment, a dose-response analysis was performed to identify differential metabolites and determine exposure levels for the second set of experiments. The second experiment included two independent treatment blocks using a single exposure level of each compound at the Cₘₐₓ or highest non-cytotoxic exposure that altered the secretome.

Training set compounds and concentrations selected for the single exposure experiments are provided in Table 1.

TABLE 1

| | | Cₘₐₓ | | Selected |
|---|---|---|---|---|
| | | Total | MAVE | Exposure |
| | Treatment | (μM) | (μM) | (μM) |
| Structural & | Amiodarone¹ | 3.9 | 3 | 3 |
| Functional | Amitriptyline¹ | 0.95 | 1 | 1 |
| Effects | Amphotericin B¹ | 89.8 | 3 | 3 |
| (“General”) | Anagrelide¹ | 0.06 | 30 | 0.6 |
| | Arsenic Trioxide¹ | 12.1 | 10 | 10 |
| | Bortezomib¹ | 0.3 | 0.1 | 0.1 |
| | Chloroquine¹ | 0.96 | 3 | 3 |
| | Clozapine¹ | 0.95 | 10 | 9.5 |
| | Dasatinib¹ | 0.72 | 10 | 0.72 |
| | Fluorouracil¹ | 4.6 | 100 | 46 |
| | Isoproterenol¹ | 0.01 | 100 | 0.1 |
| | Lapatinib¹ | 4.18 | 10 | 4.18 |
| | Mitoxantrone¹ | 3.3 | 1 | 1 |
| | Nortriptyline¹ | 0.57 | 1 | 1 |
| | Paclitaxel | 21.9 | 0.03 | 10 |
| | Sorafenib | 16.6 | 3 | 3 |
| | Sunitinib | 0.25 | 1 | 1 |
| | Vandetanib¹ | 3.3 | 1 | 1 |
| Structural | Daunorubicin¹ | 89 | 0.1 | 0.1 |
| Effects | Dexfenfluramine¹ | 0.4 | 30 | 4 |
| | Dithiazanine Iodide¹ | 0.1 | 0.1 | 0.1 |
| | Doxorubicin¹ | 15.3 | 0.1 | 0.1 |
| | Idarubicin¹ | 0.12 | 0.1 | 0.1 |
| | Imatinib¹ | 3.9 | 3 | 3 |
| | Pergolide¹ | 0.003 | 30 | 0.03 |
| | Rofecoxib¹ | 1.7 | 100 | 17 |
| | Tegaserod¹ | 0.08 | 3 | 0.08 |
| | Valdecoxib¹ | 0.51 | 100 | 5.1 |
| Functional | Astemizole¹ | 0.008 | 3 | 0.03 |
| Effects | Cisapride¹ | 0.18 | 100 | 1.8 |
| | Dofetilide¹ | 0.008 | 100 | 0.031 |
| | Encainide¹ | 0.71 | 100 | 7.1 |
| | Nifedipine¹ | 0.58 | 100 | 0.58 |
| | Sertindole¹ | 0.32 | 0.3 | 0.3 |
| | Terfenadine¹ | 0.3 | 3 | 3 |
| | Thioridazine¹ | 2.7 | 1 | 1 |
| | Verapamil | 0.815 | 3 | 0.815 |
| Non-Cardiotoxic | Acyclovir¹ | 6.7 | 100 | 6.7 |
| | Amoxicillin¹ | 17 | 100 | 17 |
| | Ascorbic Acid¹ | 36 | 100 | 36 |
| | Aspartame¹ | 1 | 100 | 1 |
| | Benzoic Acid¹ | 36 | 100 | 36 |
| | Biotin¹ | 0.01 | 100 | 0.03 |
| | Citric Acid¹ | 128 | 100 | 100 |
| | Erlotinib¹ | 3.8 | 3 | 3 |
| | Hexylresorcinol¹ | 1 | 30 | 1 |
| | Leucine¹ | 126 | 100 | 100 |
| | Maltol¹ | 30 | 100 | 30 |
| | Methapyrilene | 15.3 | 100 | 15.3 |
| | Methylparaben¹ | 0.23 | 100 | 0.23 |
| | Natamycin¹ | 0.8 | 30 | 0.8 |
| | Phenylphenol¹ | 0.09 | 100 | 0.09 |
| | Propyl Gallate | 3 | 30 | 3 |
| | Ranitidine | 1.7 | 100 | 1.7 |
| | Sorbitol | 3.9 | 100 | 3.9 |
| | Tartaric Acid¹ | 1.2 | 100 | 1.2 |
| | Thiabendazole¹ | 30.8 | 100 | 30.8 |

MAVE = Maximum Acceptable Viability Exposure (highest exposure tested with ≥90% viability).
¹Training set compounds used in Aim 4.

### Development of Data Processing Tools

To facilitate metabolomics data quality control and analysis, we developed the Toxicology Automated Processing Pipeline (TAPP) which was integrated with the Stamina Laboratory information Management System (LIMS). This pipeline included an Automated Visualization and Analysis (AVA) was incorporated into TAPP that was utilized for LC-MS quality control and exploratory data analysis as well as dose-response analysis of putative secretome biomarkers.

### Dose-Response Study

Metabolomic data was acquired to identify non-cytotoxic exposure levels and identify biomarkers of cardiotoxicity from a dose-response experiment using spent medium of iPSC-CM treated with 53 of the compounds in the training set. Briefly, human iPSC-CM (iCell® Cardiomyocytes, cat #R1007, Cellular Dynamics International) were exposed to eight concentrations of each training set compound. Spent media was collected for LC-MS analysis and cell viability was assessed with the CellTiter-Fluor Cell Viability Assay (Promega). Spent media samples were prepared and analyzed using the methods developed in Specific Aim 1. The spent media samples from iPSC-CM treated with 10 representative cardiotoxic and non-cardiotoxic compounds were analyzed with all four LC-MS methods to rank biological significant secretome features by predictive capacity. Correlation and network-based analysis of the predictive features revealed that a single LC-MS method (HILIC ESI negative) identified the metabolic profile of cardiotoxicity as well as the other three methods combined, allowing us to reduce the total number of samples analyzed and increase throughput by 75%.

Metabolic biomarkers of cardiotoxicity were ranked based on their capacity to discriminate cardiotoxic from non-cardiotoxic compounds at non-cytotoxic exposures or at exposure levels below the therapeutic Cₘₐₓ (total). Analysis of 4 class models that tested the markers ability to discriminate general, structural, functional, and non-cardiotoxic agents revealed that general cardiotoxic compounds contain a mixture of metabolic responses that resemble both functional and structural cardiotoxicity. Since general cardiotoxicants did not have a clear metabolic and toxicology distinct profile. Iwo model training regimens were used for biomarker selection and ranking, using either a 2-class (cardiotoxic vs non-cardiotoxic) or 3-class (structural, functional, and non-cardiotoxic) effect models. Exploration of the different dose-response metrics, including IC values, lowest observed effect levels (LOELs), response at Cₘₐₓ and relative overall response thresholds, demonstrated that a relative response approach was most suitable for ranking features as it was broadly applicable to single and multi-phase dose-response models. Features were ranked using random forest, support vector machines, and partial least squared discriminant analysis with 5-fold cross validation repeated 10 times with recursive feature elimination. Random forest was the best performing algorithm that identified feature sets could classify the 2-class model with 87% accuracy and the 3-class model with 76% accuracy. This analysis identified 15 secreted features that could maximize the predictive accuracy. Of these features, much of the discriminatory power resulted from four features (lactic acid, alanine, N-acetylaspartic acid, and 2′-deoxycytidine). Using the dose-response analysis of metabolomic and cell viability data, a single exposure was selected based on the lowest concentration that met the following criteria (selected exposures listed in Table 1): a) less than 10% decrease in viability; b) total Cₘₐₓ value; and c) evidence of changes in metabolism; or d) up to 10 times the total Cₘₐₓ when no evidence of metabolic changes were present.

### Single Exposure Study

The reproducibility of the putative biomarkers of cardiotoxicity identified during the dose-response study was evaluated using two replicates of the optimized single exposure levels identified through the dose-response analyses. Metabolic profiling was performed on the spent media from human iPSC-CM (iCell® Cardiomyocytes2, cat #R 1017, Cellular Dynamics International) using all four non-targeted LC-MS methods to confirm that useful markers were not missed in the dose-response analysis since a single exposure allows a more rapid execution of the training set compounds. Predictive models and feature rankings were performed using a combined set HILIC and CS features as well as each method alone. This analysis indicated that the HILIC feature set was more predictive than C8. The biomarkers measured with the HILIC LC-MS methods could predict two class toxicity (cardiotoxic vs, non-cardiotoxic) with up to 96% accuracy in the training set.

The features used in the preliminary predictive models are listed in Table 2.

TABLE 2

| | | | | | |
|---|---|---|---|---|---|
| | | | | Retention | LC-MS-MS |
| | Aim 2 | | | Time | Confirmation |
| Metabolite Name | Unique ID | Adduct | m/z | (Seconds) | (Y/N) |

HILIC ESI negative features

| | | | | | |
|---|---|---|---|---|---|
| Arachidonic acid | qc0286 | [M − H]⁻ | 303.2332 | 55 | Y |
| Thymidine | qc0331 | [M + Cl]⁻ | 277.0618 | 132 | Y |
| Pyruvate | qc0336 | [M − H]⁻ | 87.0088 | 217 | Y |
| Pyruvate | qc0342 | [M − H]⁻ | 87.0092 | 236 | Y |
| 2′-deoxycytidine | qc0344 | [M + Cl]⁻ | 262.0615 | 296 | Y |
| Inosine | qc0345 | [M − H]⁻ | 267.0742 | 318 | Y |
| Lactic acid | qc0357 | [M − H]⁻ | 89.0244 | 373 | Y |
| Alanine | qc0379 | [M − H]⁻ | 88.0404 | 572 | Y |
| N-acetylaspartic acid | qc0390 | [M − H]⁻ | 174.0410 | 695 | Y |
| N/A | qc0291 | N/A | 281.2482 | 55 | N |
| N/A | qc0585 | N/A | 651.4394 | 56 | N |
| N/A | qc0601 | N/A | 385.1921 | 56 | N |
| N/A | qc0296 | N/A | 337.1945 | 56 | N |
| N/A | qc0298 | N/A | 253.2167 | 57 | N |
| N/A | qc0304 | N/A | 251.2004 | 58 | N |
| N/A | qc0664 | N/A | 225.1849 | 58 | N |
| N/A | qc0306 | N/A | 223.1697 | 59 | N |
| N/A | qc0655 | N/A | 249.1840 | 59 | N |
| N/A | qc0322 | N/A | 229.1449 | 67 | N |
| N/A | qc0327 | N/A | 307.1031 | 120 | N |
| N/A | qc0625 | N/A | 306.0598 | 229 | N |
| N/A | qc0668 | N/A | 187.0412 | 374 | N |
| N/A | qc0647 | N/A | 271.1021 | 454 | N |
| N/A | qc0291 | N/A | 281.2482 | 55 | N |

C8 ESI negative features

| | | | | | |
|---|---|---|---|---|---|
| N/A | qc0833 | N/A | 1124.0009 | 87 | N |
| N/A | qc0810 | N/A | 181.0521 | 166 | N |
| N/A | qc0060 | N/A | 229.1447 | 453 | N |
| N/A | qc0766 | N/A | 315.2555 | 473 | N |
| N/A | qc0085 | N/A | 293.2133 | 540 | N |
| N/A | qc0091 | N/A | 249.1871 | 575 | N |
| N/A | qc0786 | N/A | 251.2030 | 614 | N |

A predictive model was created from the structurally confirmed biomarkers (see Specific Aim 3 results) using the two replications of the single exposure training set and tested against a blinded set of 12 compounds not utilized in the training of the predictive model (Table 3).

Test set results are provided in Table 3.

TABLE 3

| Non-Cardiotoxicants | | | | | Cardiotoxicants | | | | | |
|---|---|---|---|---|---|---|---|---|---|---|
| | Cmax | | Global | Functional | | | Cmax | | Global | Functional |
| | Total | Exposure | Tox Model | Model | | Type of | Total | Exposure | Tox Model | Model |
| Treatment | (μM) | (μM) | Prediction | Prediction | Treatment | Cardiotoxicity | (μM) | (μM) | Prediction | Prediction |
| Acetylsalicylic | 10 | 10 | − | − | Bepridil | Functional | 3.3 | 3.3 | + | + |
| Acid | | | | | | | | | | |
| Cetirizine | 0.8 | 0.8 | − | − | Busulfan | Structural | 50 | 150 | + | − |
| Loratadine | 0.02 | 0.02 | − | − | Levomethadyl | Functional | 0.6 | 6 | + | + |
| | | | | | Acetate | | | | | |
| Sildenafil | 1 | 1 | − | − | Nilotinib | Functional & | 3 | 3 | + | − |
| | | | | | | Structural | | | | |
| Sucrose | 1.8 | 1.8 | − | − | Rosiglitazone | Structural | 1.7 | 17 | − | − |
| Xylitol | 0.5 | 0.5 | − | − | Sotalol | Functional | 15 | 45 | + | + |

−: Compound predicted to be negative in specified model.
+: Compound predicted to be positive (cardiotoxic or functional) in specified model.

The models generated from three confirmed metabolites and viability could predict the training set with 88% accuracy (89% sensitivity, 85% specificity) and the test set was predicted with 92% accuracy (83% sensitivity, and 100% specificity). A second predictive model was created to distinguish general or structural cardiotoxicants from functional cardiotoxicants, which identified functional cardiotoxicants with 95% accuracy in the training set and 100% accuracy in the test set.

Specific Aim 3: Confirm the structural identity of the predictive metabolites and evaluate their biological significance as confirmed biomarkers.

The objective of Aim 3 was to confirm the identity of the predictive metabolites that were reproducibly identified in Aim 2. There were 23 predictive features identified in the HILIC ESI negative LC-MS method and 7 predictive features in the C8 ESI negative LC-MS method that had unknown chemical structures (Table 2). The features in the HILIC ESI negative LC-MS method were selected for structure confirmation by LC-MS-MS analysis due to the larger number of features and inclusion in more predictive models than the features identified in the C8 ESI negative method. Chemical structure identification was conducted by acquiring LC-MS-MS data for reference compounds and comparing the spectra to LC-MS-MS data of the predictive features from samples collected in Aim 2 testing. Reference compounds were purchased and spiked into iCell Cardiomyocyte Maintenance medium and prepared the using the same protocol for spent media samples collected in Aim 2. The LC-MS-MS spectra data was collected using Collision Energies of 10, 20, and 40 V. Both the retention time and the LC-MS-MS spectra had to be a reasonable match between the discovery sample and the spiked in reference sample. After the structural identification process was completed, 9 out of the 23 HILIC ESI negative features were able to be confirmed.

Specific Aim 4: Develop a targeted biomarker assay for the metabolites predictive of cardiotoxicity and evaluate the productivity using the targeted methods.

The goal of Aim 4 is to develop a targeted LC-MS method with increased throughput to analyze predictive metabolites that were confirmed in Aim 3. The sample preparation method was optimized to be used in a normal phase LC-MS method and stable labeled internal standards (L-Citrulline-D4. Thymidine-D4, and L-Lactic acid-D3) were added for quality control and normalization. Different combinations of HILIC UPLC columns and mobile phases were screened to identify the best chromatographic conditions for the confirmed metabolites (Table 4).

Variables in high throughput HILIC negative chromatography method development screening are provided in Table 4.

TABLE 4

| UPLC HILIC Column | Mobile | Chromatography Notes |
|---|---|---|
| Waters, Acquity UPLC BEH | Low pH¹ | Acceptable peak shape for 6/7 predictive markers |
| Amide 1.7 μm, 2.1 × 50 mm | Middle pH² | Acceptable peak shape for 7/7 predictive markers |
| Part No.: 186004800 | High pH³ | Acceptable peak shape for 5/7 predictive markers |
| Phenomenex, Kinetex | Low pH¹ | Acceptable peak shape for 4/7 predictive markers |
| HILIC 1.7 μm, 2.1 × 50 | Middle pH² | Acceptable peak shape for 3/7 predictive markers |
| mm Part No.: 00B-4474-AN | High pH³ | Acceptable peak shape for 4/7 predictive markers |
| Phenomenex, Luna | Low pH¹ | Acceptable peak shape for 2/7 predictive markers |
| NH2 3 μm, 50 × 2 | Middle pH² | Acceptable peak shape for 5/7 predictive markers |
| mm Part No.: 00B-4377-B0 | High pH³ | Acceptable peak shape for 4/7 predictive markers |

¹Mobile Phase A = 10 mM Ammonium Formate and 0.125% Formic Acid in Water; Mobile Phase B = 10 mM Ammonium Formate and 0.125% Formic Acid in ACN:Water (90:10).
²Mobile Phase A = 5 mM Ammonium Acetate pH 4.7 in Water; Mobile Phase B = 5 mM Ammonium Acetate pH 4.7 in ACN:Water (90:10).
³Mobile Phase A = 10 mM Ammonium Acetate and 0.04% Ammonium Hydroxide in Water; Mobile Phase B = 10 mM Ammonium Acetate and 0.04% Ammonium Hydroxide in ACN:Water (90:10).

Chromatography evaluations were based on 7 structurally confirmed makers that were important for building predictive models of cardiotoxicity (Table 2).

The Waters. Acquity UPLC BEH Amide column with the middle pH mobile phase provided the best peak shape for markers of cardiotoxicity. This targeted LC-MS method has been tested with the training and blinded test set compounds (over 80 compounds total) to evaluate the reproducibility of the method as well as assay performance.

We analyzed a training set of 49 compounds (see Table 1) to develop a commercially relevant predictive model of cardiotoxicity based on changes in iPSC-CM metabolism. The individual metabolites measured by the rapid LC-MS method and combinations of these metabolites along with viability were evaluated for their capacity to discriminate cardiotoxic from non-cardiotoxic compounds. Three different normalization methodologies were also included in the analysis to identify the most predictive combination of metabolites and normalization methods. The response at the Cₘₐₓ exposure level for each compound was used to score classification performance. The scoring algorithm was: For each compound if a feature exhibits a response below the compounds Cₘₐₓ value than it is scored as cardiotoxic and if a feature exhibits a response above the Cₘₐₓ value it is scored as non-cardiotoxic. These scores are compared to the known toxicities of the training set compounds to determine performance using confusion matrix-derived metrics. Each metabolite or metabolite combination was evaluated to identify a response level that could maximally discriminate cardiotoxic from non-cardiotoxic compound with a high positive predictive value. The best performing commercial candidate predictive model of cardiotoxicity utilized a ratio of lactic acid and arachidonic acid normalized to viability.

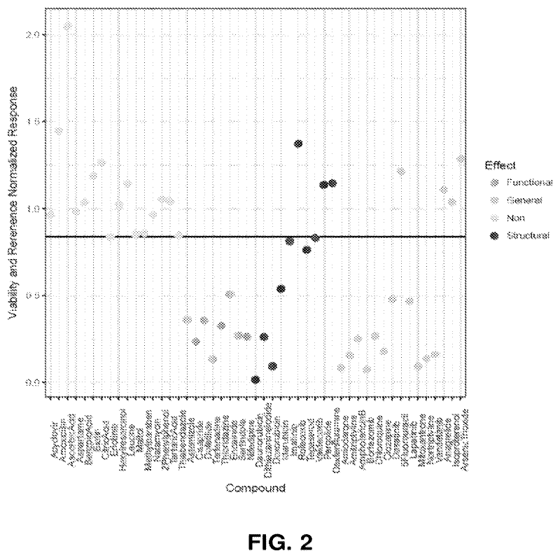

FIG. 2 is a graph showing the interpolated response at the Cₘₐₓ classification exposure level from the dose-response model fit of a ratio of the fold changes of lactic acid and arachidonic acid normalized to viability. The horizontal line indicates the response threshold that can maximally discriminate cardiotoxic from non-cardiotoxic compounds. Compounds are colored by type of toxicity.

In this simplified predictive model (FIG. 2) an interpolated response of less than 0.84 at the Cₘₐₓ concentration had an optimal discriminatory power to identify cardiotoxicants with a high positive predictive value. This model was able to classify the training set of compounds with an accuracy of 86%, a specificity of 100%, sensitivity of 79%, positive predictive value of 100% and a negative predictive value of 70% with an AUC of 0.84.

The ratio of lactic acid and arachidonic acid is able to identify all the functional cardiotoxicants, 70% of the structural cardiotoxicants, 75% of the general cardiotoxicants without misclassifying a single non-cardiotoxicants. The predictive capacity of this model as well as the throughput to measure the metabolites meets our requirements to enter commercialization and move into the product development pipeline.

### Example 3

### In Vitro Assay to Predict Cardiotoxicity Potential Using Targeted Metabolomics and Human Induced Pluripotent Stem Cell-Derived Cardiomyocytes

Cardiac safety one of the leading causes of late-stage compound attrition in the pharmaceutical industry and accounts for 23% of the safety related withdrawals of FDA-approved drugs from the market.

Currently, preclinical evaluation of cardiac safety is heavily focused on approximately 3-7 main ion channels involved m maintaining the cardiac action potential; however, over 70 different types of ion channels are expressed in the heart and participate in the overall cardiac function. These safety testing methods overemphasize electrophysiological assessment of cardiotoxicity and fail to evaluate cardiomyopathy and other forms of structural cardiotoxicity.

Metabolic perturbations are one-of the primary mechanisms of cardiotoxicity elicited by pharmaceuticals.

We have developed a biomarker-based essay for predicting the cardiotoxicity potential of compounds based on changes in metabolism as observed through the secretome human induced pluripotent stem sell (hiPSC)-derived cardiomyocytes.

In this study, we exposed hiPSC-derived cardiomyocytes to a training set of 57 compounds (37 positive, 20 negative) and analyzed the spent media using untargeted UPLC-HRMS-based metabolomics. The cardiotoxic compounds were broken into three categories, structural, functional, and general (compounds that cause both). Analysis of metabolomics data identified a set of biomarkers that represent different metabolic pathways.

A rapid, targeted UPLC-HRMS method was developed for the most predictive biomarkers. The individual metabolites and combinations of these metabolites along with viability were evaluated for their capacity to discriminate cardiotoxic from non-cardiotoxic compounds. The predictive models were evaluated: with a blinded test set of 24 compounds (12 positive, 12 negative).

This assay is an attractive new model that can identify both structural and functional cardiotoxic compounds that could be used in conjunction with CiPA and other endpoints to provide a comprehensive evaluation of a compound's cardiotoxicity potential.

Human induced pluripotent stem (iPS) cell derived cardiomyocytes (Cellular Dynamics International. Inc.) were plated in 96-well plates and cultured for 4 days, allowing the cells to form electrically connected syncytial layers that beat in synchrony.

Cells were exposed to test article for 72 hours. Media±test article were replaced approximately every 24 hours.

Spent media from the last 24-hour treatment period was collected. Proteins were precipitated and removed using a methanol and acetonitrile solution.

Cell viability was assess after sample collection using the CII Titler-Fluor Cell Viability Assay (Promega).

Samples were analyzed with:
Phase 1: 4 non-targeted HILIC chromatography coupled to electrospray ionization in both positive and negative ion polarities. The identity of the most predictive metabolites was confirmed by LC-MS-MS.
Phase 2: a rapid UPLC-HRMS method optimized for the predictive metabolites.

Phase 1: Metabolite biomarkers of cardiotoxicity were ranked base on their capacity to discriminate cardiotoxic from non-cardiotoxic exposures or at exposure levels below the therapeutic Cₘₐₓ (total). Features were ranked using random forest, support vector machines, and partial least squared discriminant analysis with 5-fold cross validation repeated 10 times with recursive feature elimination.

Phase 2: The individual metabolites measured by the rapid LC-MS method and combinations of these metabolites along with viability were evaluated for their capacity to discriminate cardiotoxic from non-cardiotoxic compounds. The response at the Cₘₐₓ, 3×Cₘₐₓ, and 10×Cₘₐₓ exposure levels for each compound was used to score classification performance.

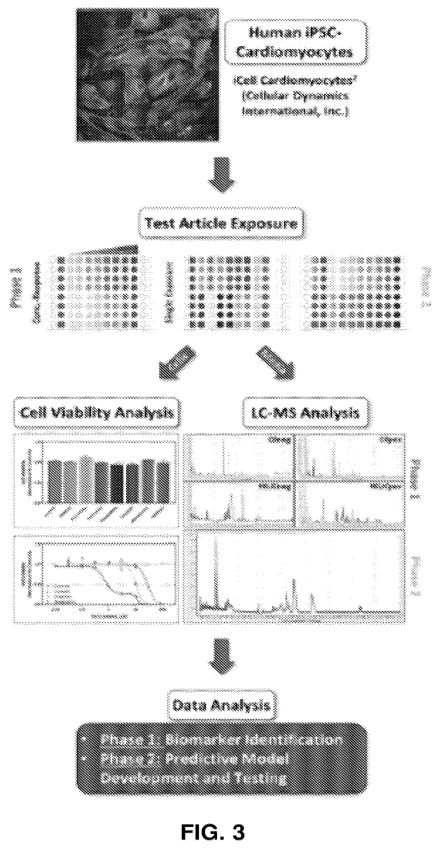

FIG. 3 is a schematic of the method used to evaluate cardiotoxicity of compounds.

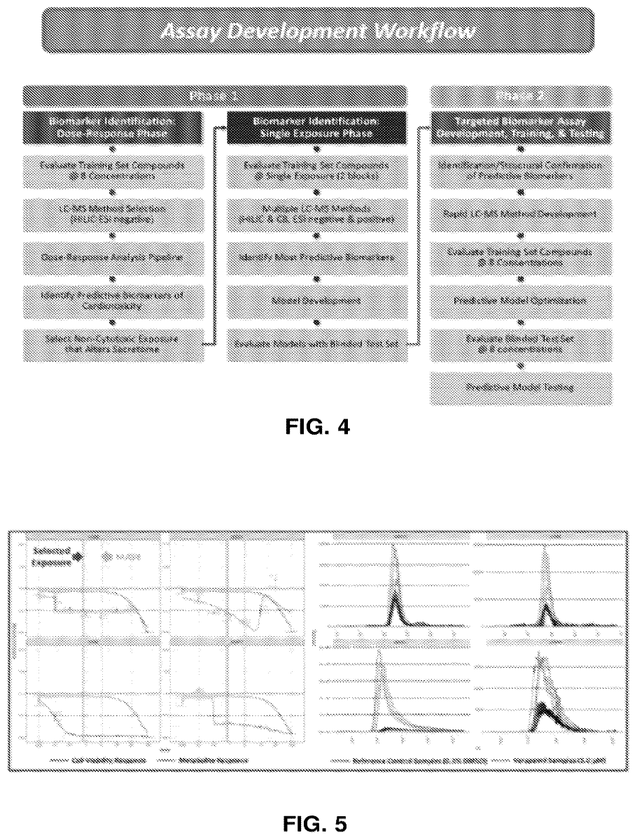

FIG. 4 is a schematic of the assay development workflow used to evaluate cardiotoxicity of compounds.

The goal of the concentration-response phase of biomarker identification was to identify non-cytotoxic exposure levels that change iPSC-CM metabolism. The method included modeling feature response, calculating maximum acceptable viability (MAVE), determining response at Cₘₐₓ, and MAVE, reviewing the top 4 responding features, and selecting the highest tested exposure with 90% viability.

FIG. 5 shows graphs from results of the concentration-response phase of biomarker identification. Panels on the left are graphs of cell viability response and metabolite response, as indicated. Panels on the right are graphs of reference control samples and verapamil samples, as indicated.

Exposure selection information is provided in Table 5.

TABLE 5

| Treatment | Verapamil |
|---|---|
| Effect | Functional |
| Cₘₐₓ Total (μM) | 0.815 |
| Cₘₐₓ Free | 0.07 |
| MAVE (μM) | 3 |
| Selected Exposure (μM) | 0.815 |

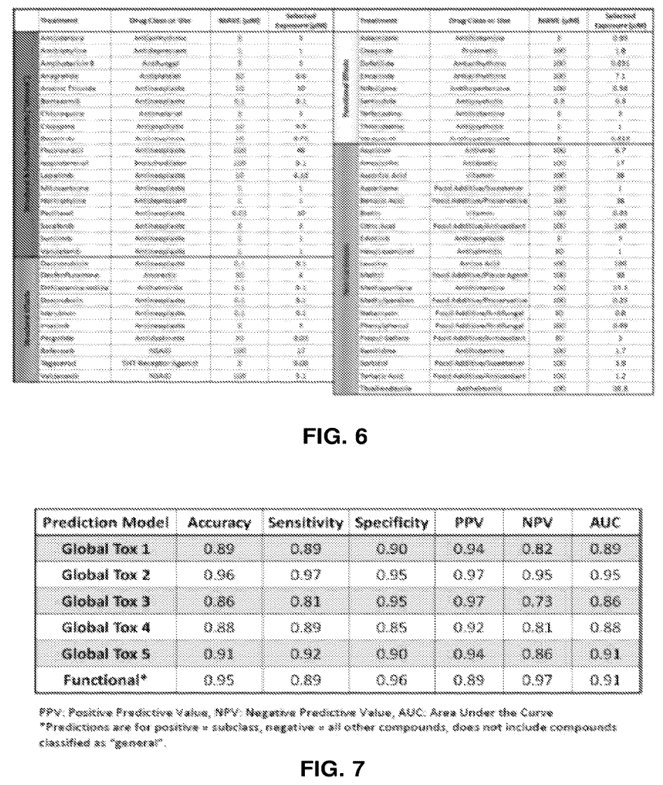

FIG. 6 is a table showing exposure levels selected for single exposure phase.

The single exposure phase of the study was used to refine biomarker profiles and evaluate the reproducibility using non-cytotoxic single exposure levels. Multiple models were generated using combinations of individual metabolites, metabolite ratios, and cell viability.

FIG. 7 is a table showing training set results from the single exposure phase. Sensitivity indicates detection of cardiotoxic compounds. Specificity indicates detection of non-cardiotoxic compounds. PPV is the percent of compounds predicted to be cardiotoxic that are true cardiotoxicants. NPV is the percent of compounds predicted to be non-cardiotoxic that are true non-cardiotoxic compounds.

Prediction models were evaluated in an independent test set of 12 compounds evaluated at 3 concentrations to verify the reproducibility and accuracy of the biomarkers prior to moving forward with targeted assay development.

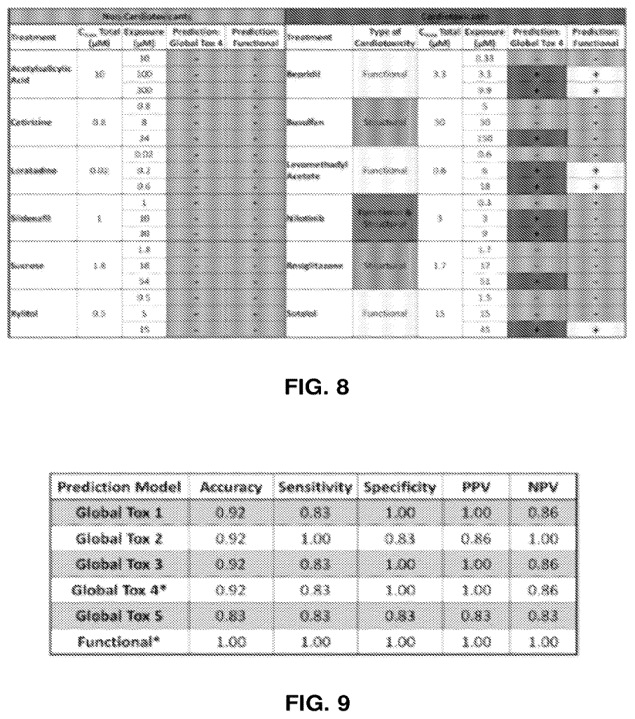

FIG. 8 is a table of non-cardiotoxicants and cardiotoxicants identified in the single exposure phase.

FIG. 9 is a table showing characteristics of various global toxicity models. Model accuracy was based on correct classification at ≤10-fold of the therapeutic total Cₘₐₓ.

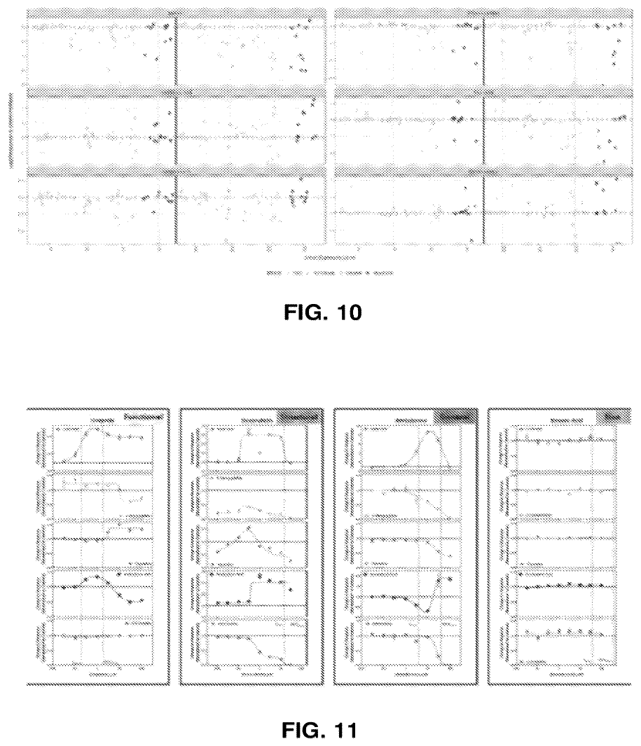

FIG. 10 shows training set results for individual metabolites and metabolite condition utilized in the Cardio quickPredict assay at the therapeutic total Cₘₐₓ, and 10×Cₘₐₓ.

FIG. 11 Shows graphs of the metabolite responses for a subset of the training set compounds. The concentration-response curves for biomarker ratios vary between types and mechanisms of cardiotoxicity.

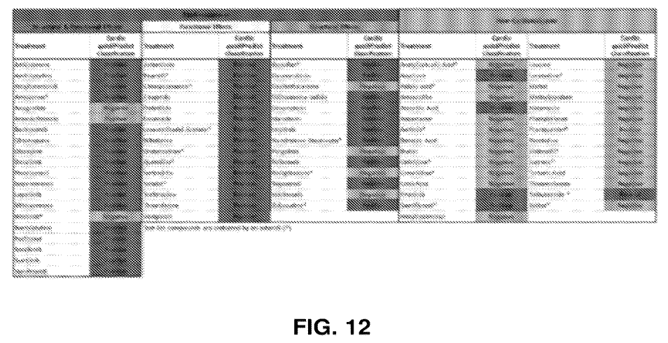

FIG. 12 is a table of compounds tested in the single exposure phase and whether they were predicted to be cardiotoxicants by Cardio quickPredict.

Prediction data is summarized in Table 6.

TABLE 6

| Compound | Balanced | | | | |
|---|---|---|---|---|---|
| Set | Accuracy | Sensitivity | Specificity | PPV | NPV |
| Training Set | 86% | 89% | 82% | 92% | 78% |
| Test Set | 79% | 83% | 75% | 77% | 82% |
| Combined | 84% | 88% | 79% | 88% | 79% |

* Combination of 4 metabolites was able to classify the training set with 86% accuracy and the test set with 79% accuracy, for a combined accuracy of 84% across 79 compounds.

Exposure to cardiotoxic compounds with varying mechanisms of toxicity alters human iPSC-derived cardiomyocyte metabolism.

Metabolites selected for the final model exhibited a reproducible response indicative of cardiotoxicity in three independent experiments.

Metabolite reties detect cardiotoxicity potential independent of changes in cell viability.

This method cars be combined with other assays, or endpoints for a comprehensive understanding of a compound's cardiotoxicity liability.

### Example 4

### Metabolites

Using the methodologies described in Examples 1 to 3, the following metabolites associated with cardiotoxicity have been identified lactic acid, thymidine, arachidonic acid, 2′-deoxycytidine, and N-acetylaspartic acid. Using the methodologies described in Examples 1 and 2, the additional metabolites associated with cardiotoxicity have been identified as alanine, pyruvate, and inosine.

### Example 5

### Ratios

Using the methodologies described in Examples 1 to 3, a model that combines lactic acid, 2′-deoxycytidine (2dC), thymidine, and arachidonic acid has been formulated that is predictive of cardiotoxicity.

Further, the following ratios of metabolites have been identified as predictive of cardiotoxicity.

Ratios that are Predictive:

Arachidonic Acid/Lactic Acid
2′-deoxycytidine/Thymidine
Lactic Acid/Thymidine
N-acetylaspartate/2dC
Thymidine to Arachidonic acid

And, various pairings of these ratios, as set forth below, have been identified as predictive of cardiotoxicity.

Pairs of ratios that are predictive:

| Ratio 1 | Ratio 2 |
|---|---|
| Lactic Acid/Thymidine | N-acetylaspartate/2dC |
| Lactic Acid/Thymidine | 2′-deoxycytidine/Thymidine |
| Arachidonic Acid/Lactic Acid | N-acetylaspartate/2dC |
| Arachidonic Acid/Lactic Acid | 2′-deoxycytidine/Thymidine |
| Thymidine/Arachidonic Acid | Lactic Acid/Thymidine |
| Thymidine/Arachidonic Acid | N-acetylaspartate/2dC |
| Thymidine/Arachidonic Acid | 2′-deoxycytidine/Thymidine |
| Thymidine/Arachidonic Acid | Arachidonic Acid/Lactic Acid |

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of assessing cardiotoxicity of a test compound, the method comprising:
contacting an in vitro culture of human cardiomyocyte cells with a test compound:
assaying the spent cell culture media for one or mom metabolites selected from the group consisting of arachidonic acid, thymidine, pyruvate, 2′-deoxycytidine, inosine, lactic acid, alanine, and N-acetylaspartic acid,
wherein a statistically significant abundance difference as compared to cardiomyocyte controls in the one or more of the metabolites indicates cardiotoxicity.

2. The method of claim 1, comprising removing proteins from the sample of the spent cell culture media prior to assaying a sample of the spent cell culture media for one or more of the metabolites.

3. The method of claim 2, wherein removing proteins from the sample of the spent cell culture media comprises removing proteins by precipitation.

4. The method of claim 3, comprising precipitation at about 50% methanol, about 35% acetonitrile, and about 15% sample.

5. The method of claim 1, comprising assaying for two or more of the metabolites.

6. The method of claim 1, comprising assaying for three or more of the metabolites.

7. The method of claim 1, comprising assaying for lactic acid, thymidine, arachidonic acid, 2′-deoxycytidine, and N-acetylaspartic acid.

8. The method of claim 1, comprising assaying for alanine, pyruvate, and inosine.

9. The method of any one of claims 1 to 8, further comprising an assessment of the relative abundance of two or more of the metabolites.

10. The method of any one of claims 1 to 9, further comprising determining a ratio of two or more of the metabolites.

11. The method of any one of claims 1 to 10, comprising assaying for:
lactic acid and arachidonic acid;
2′-deoxycytidine and thymidine;
lactic acid and thymidine;
N-acetylaspartate and 2*-deoxycytidine; and/or
thymidine to arachidonic acid.

12. The method of claim 11, further comprising determining the ratio of:
lactic acid to arachidonic acid;
2′-deoxycytidine to thymidine;
lactic acid to thymidine;
N-acetylaspartate and 2′-deoxycytidine; and/or
thymidine to arachidonic acid.

13. The method of claim 12, wherein the ratio of lactic acid to arachidonic acid, 2′-deoxycytidine to thymidine, lactic acid to thymidine, N-acetylaspartate to 2′-deoxycytidine, and/or thymidine to arachidonic acid is indicative of cardiotoxicity.

14. The method of claim 12, wherein a ratio of lactic acid to arachidonic acid of about 0.84 or less is indicative of cardiotoxicity.

15. The method of any one of claims 1 to 9, further comprising determining two or more ratios of the metabolites.

16. The method of claim 15 comprising determining the ratios of:
lactic acid to thymidine and N-acetylaspartate to 2′-deoxycytidine;
lactic acid to thymidine and 2′-deoxycytidine to thymidine;
arachidonic acid to lactic acid and N-acetylaspartate to 2′-deoxycytidine;
arachidonic acid to lactic acid and 2′-deoxycytidine to thymidine;
thymidine to arachidonic acid and lactic acid to thymidine;
thymidine to arachidonic acid and N-acetylaspartate to 2′-deoxycytidine;
thymidine to arachidonic acid and 2′deoxycytidine to thymidine; and/or
thymidine to arachidonic acid and arachidonic acid to lactic acid

17. The method of claim 16, wherein one or more pairings of ratios is indicative of cardiotoxicity.

18. A method comprising:
contacting an in vitro culture of human cardiomyocyte cells with a test compound; and
assaying the spent cell culture media for one or more metabolites selected from the group consisting of arachidonic acid, thymidine, pyruvate, 2′-deoxycytidine, inosine, lactic acid, alanine, and N-acetylaspartic acid.

19. The method of claim 18, comprising removing proteins from the sample of the spent cell culture media prior to assaying a sample of the spent cell culture media for the one or more metabolites.

20. The method of claim 19, wherein removing proteins from the sample of the spent cell culture media comprises removing proteins by precipitation.

21. The method of claim 20, comprising precipitation at about 50% methanol, about 35% acetonitrile, and about 15% sample.

22. The method of claim 18, assaying for two or more of the metabolites.

23. The method of claim 18, comprising assaying for three or more of the metabolites.

24. The method of claim 18, comprising assaying for lactic acid, thymidine, arachidonic acid, 2′-deoxycytidine, and N-acetylaspartic acid.

25. The method of claim 18, comprising assaying for alanine, pyruvate, and inosine.

26. The method of any one of claims 18 to 25, further comprising an assessment of the relative abundance of two or more of the metabolites.

27. The method of any one of claims 18 to 26, further comprising determining a ratio of two or more of the metabolites.

28. The method of any one of claims 18 to 27, comprising assaying for:
lactic acid and arachidonic acid;
2′-deoxycytidine and thymidine;
lactic acid and thymidine;
N-acetylaspartate and 2′-deoxycytidine; and/or
thymidine to arachidonic acid.

29. The method of claim 28, further comprising determining the ratio of:
lactic acid and arachidonic acid;
2′-deoxycytidine and thymidine;
lactic acid and thymidine;
N-acetylaspartate and 2′-deoxycytidine; and/or
thymidine to arachidonic acid.

30. The method of any one of claims 18 to 2, further comprising determining two or more ratios of the metabolites.

31. The method of claim 30 comprising determining the ratios of:
lactic acid to thymidine and N-acetylaspartate to 2′-deoxycytidine;
lactic acid to thymidine and 2′-deoxycytidine to thymidine;
arachidonic acid to lactic acid and N-acetylaspartate to 2′-deoxycytidine;
arachidonic acid to lactic acid and 2′-deoxycytidine to thymidine;
thymidine to arachidonic acid and lactic acid to thymidine;
thymidine to arachidonic acid and N-acetylaspartate to 2′-deoxycytidine;
thymidine to arachidonic acid and 2′deoxycytidine to thymidine; and/or
thymidine to arachidonic acid and arachidonic acid to lactic acid

32. The method of any one of claims 1 to 31, wherein the one or more metabolites are assayed by a physical separation method.

33. The method of claim 32, wherein the physical separation method comprises liquid chromatography mass spectrometry (LC-MS).

34. The method of claim 32, physical separation comprises one or more methodologies selected from CS liquid chromatography coupled to electrospray ionization in positive ion polarity (C8pos), C8 liquid chromatography coupled to electrospray ionization in negative ion polarity (C8neg), hydrophilic interaction liquid chromatography coupled to electrospray ionization in positive ion polarity (HILICpos), and/or hydrophilic interaction liquid chromatography coupled to electrospray ionization in negative ion polarity (HILICneg).

35. The method of any one of claims 1 to 31, wherein the one or more metabolites are assayed using a colorimetric or immunological assay.

36. The method of any one of claims 1 to 35, wherein the cardiomyocytes are cultured at a concentration of the test compound comprising the test compound's human therapeutic Cₘₐₓ.

37. The method of any one of claims 1 to 36, wherein the human cardiomyocyte cells comprise human induced pluripotent stem cell (hiPSC)-derived cardiomyocyte cells.

38. A method of assessing cardiotoxicity of a test compound as described herein.

39. A metabolomic signature for cardiotoxicity, the metabolomic signature comprising two or more metabolites selected from the group consisting of arachidonic acid, thymidine, pyruvate, 2′-deoxycytidine, inosine, lactic acid, alanine, and N-acetylaspartic acid.

40. A metabolomic signature for cardiotoxicity as described herein.
